(19) United States
(12) Patent Application Publication
(10) Pub. No.: US 2020/0390506 A1
(43) Pub. Date: Dec. 17, 2020

(54) WORKFLOW CONTROL WITH TRACKED DEVICES

(71) Applicant: Think Surgical, Inc., Fremont, CA (US)

(72) Inventor: Daniel Patrick Bonny, Fremont, CA (US)

(73) Assignee: Think Surgical, Inc., Fremont, CA (US)

(21) Appl. No.: 16/978,370

(22) PCT Filed: Mar. 7, 2019
(86) PCT No.: PCT/US2019/021166

§ 371 Date: Sep. 4, 2020

Related U.S. Application Data
(60) Provisional application No. 62/639,672, filed on Mar. 7, 2018.

(51) Int. Cl. A61B 34/20; A61B 34/00; A61B 34/10; A61B 90/00

(52) U.S. Cl. CPC A61B 34/20; A61B 34/25; A61B 34/10; A61B 2090/3945; A61B 2034/2055; A61B 2034/252; A61B 2034/254; A61B 90/39

(57) ABSTRACT

A method and system for controlling and executing a workflow during a computer-assisted surgical procedure include providing an optical tracking system having a field of view and being in communication with the workflow, introducing a first tracked device into the field of view, identifying the first tracked device with the tracking system based on a first reference member associated with the first tracked device, determining a first step in the workflow based on the identification of the first tracked device, and displaying the first step to a user on a graphical user interface. The optical tracking system having a processor with software executable instructions for identifying the presence or absence of either the tracked implements in the field of view of the tracking system, determining a workflow step based on identification of the tracked implement, and commanding the workflow to display the determined step on the graphical user interface.

### RELATED APPLICATIONS

This application claims priority benefit of U.S. Provisional Application Ser. No. 62/639,672 filed 7 Mar. 2018, the contents of which are hereby incorporated by references.

### TECHNICAL FIELD

The present invention generally relates to the field of computer-assisted surgery, and more particularly to a system and method for controlling a surgical workflow with tracked devices.

### BACKGROUND

A surgical workflow consists of a plurality of steps that guides a surgical team through a surgical procedure. Each of the plurality of steps provides instructions to the user to complete a particular action for that step, or in some cases simply convey information to the user. The instructions or information is typically displayed on a monitor in the operating room (OR) in the form of text or graphics, or in some instances provided in audible or tactile form. The workflow may further include a plurality of options or functions that allow a user to perform additional tasks or repeat one or more of the surgical steps. For example, a workflow for a robotic-assisted total knee arthroplasty (TKA) may consist of the following steps. A first screen displaying, “Prepare the patient by making an incision on the skin and exposing the distal femur and proximal tibia.” After user confirmation, a second screen displays, “Register the femur and the tibia.” Once successfully registered, a third screen displays, “Guide the robotic arm to the top of the bone.” Once the arm is positioned, a fourth screen displays, “Ready to cut?” And once confirmed, the robotic arm cuts the femur and tibia to receive an implant according to a pre-operative surgical plan.

As a surgical workflow is absolutely necessary to complete a successful computer-assisted surgery, the current methods of controlling the workflow are time consuming, cumbersome, and require a steep learning curve. For instance, many workflows require the use of a controller or pendant to permit the user to interact and control the workflow (e.g., confirm a step, navigate to an options menu, collect a registration point). The controllers are hard wired to the computer-assist device as a safety measure, but at the expense of having an additional wire cluttering the workspace. As a workaround, the display monitor may include a touch screen. However, depending on the location of the monitor in the OR, the ability to interact and control the workflow with a touchscreen monitor may be difficult, or at least require a dedicated surgical team member to be in proximity of the monitor. In addition, the current controllers generally include several buttons for navigating through the workflow. As the number of buttons increase, the versatility of controlling the workflow increase. However, the learning curve also increases as well as the odds of activating or pressing an incorrect button.

Thus, there exists a need for a system and method to more efficiently and effectively control a workflow to complete a computer-assisted surgical procedure.

### SUMMARY

A method for controlling a workflow during a computer-assisted surgical procedure is provided that includes providing an optical tracking system having a field of view of a surgical site and in communication with the workflow, introducing a first tracked device into the field of view, identifying the first tracked device with the tracking system based on a first reference member associated with the first tracked device, determining a first step in the workflow based on the identification of the first tracked device, and displaying the first step to a user on a graphical user interface.

A computer-assisted surgical system for executing the method for controlling a workflow during a computer-assisted surgical procedure is also provided that includes an optical tracking system having a processor with software executable instructions for identifying the presence or absence of either a tracked digitizer probe or a tracked surgical device in the field of view of the tracking system, determine a step in the workflow based on the identification of either the tracked digitizer probe or tracked surgical device, and command the workflow to display the determined step on the graphical user interface.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 depicts a surgical workflow in accordance with embodiments of the invention;

FIG. 2 depicts a registration workflow in accordance with embodiments of the invention;

FIGS. 3A-3D depict several surgery mode workflows in accordance with embodiments of the invention, where FIG. 3A depicts a first surgery mode, FIG. 3B depicts a second surgery mode. FIG. 3C depicts a third surgery mode, and FIG. 3D depicts a fourth surgery mode;

FIG. 4 depicts a surgical system implementing the workflow of FIG. 1 in accordance with embodiments of the invention;

FIG. 5 depicts a tracked surgical device in accordance with embodiments of the invention; and

FIG. 6 depicts a tracked digitizer probe in accordance with embodiments of the invention.

### DETAILED DESCRIPTION

The present invention has utility as a system and method to control a workflow to for a computer-assisted surgical procedure with superior efficiency than is presently possible. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Further, it should be appreciated that although the systems and methods described herein make reference to total knee arthroplasty, the systems and methods may be applied to other computer-assisted surgical procedures involving other bones and joints in the body illustratively including the hip, ankle, elbow, wrist, skull, and spine, as well as partial replacement procedures and the revision of initial repair or replacement of any of the aforementioned bones or joints.

Additionally, it should be appreciated that the embodiments of a system and method to efficiently and effectively control a workflow for a computer-assisted surgical procedure disclosed herein do not preempt the field of workflow control for computer-assisted surgical procedures.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “pre-operative bone data” refers to bone data used to pre-operatively plan a procedure before making modifications to the actual bone. The pre-operative bone data may include one or more of the following: an image data set of a bone (e.g., acquired via computed tomography (CT), magnetic resonance imaging (MRI), ultrasound, x-ray, laser scan, etc.), a virtual generic bone model, a physical bone model, a virtual patient-specific bone model generated from an image data set of a bone, a set of data collected directly on a bone intra-operatively (commonly used with imageless computer-assist devices), etc.

As used herein, the term “registration” refers to the determination of the position and orientation (POSE) and/or coordinate transformation between two or more objects or coordinate systems such as a computer-assist device, a bone, pre-operative bone data, surgical planning data (i.e., an implant model, cut-file, virtual boundaries, virtual planes, cutting parameters associated with or defined relative to the pre-operative bone data), and any external landmarks (e.g., a tracking array) associated with the bone, if such landmarks exist. Conventional methods of registration known in the art are described in U.S. Pat. Nos. 6,033,415; 8,010,177; and 8,287,522.

As used herein, the term “real-time” refers to the processing of input data within milliseconds such that calculated values are available within 10 seconds of computational initiation.

Also used herein is the term “optical communication” which refers to wireless data transfer via infrared or visible light as described in U.S. patent application Ser. No. 15/505,167 assigned to the assignee of the present application and incorporated by reference herein in its entirety.

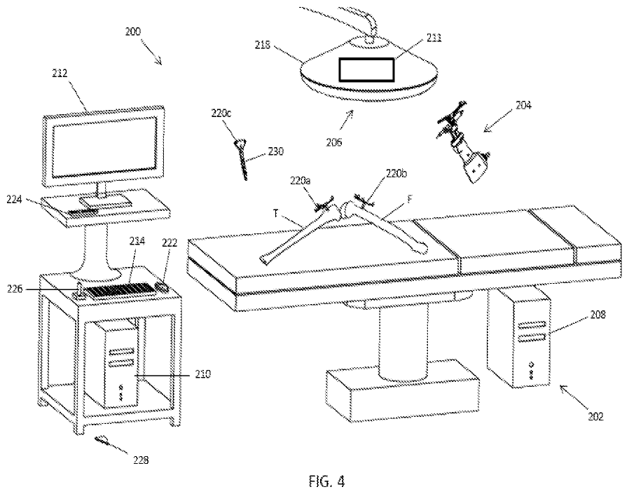

Further, the following description makes reference to computer-assisted surgical systems to perform the computer-assisted surgical procedures, in which an example of a computer-assisted surgical system 200 is described in detail below with reference to FIGS. 4-6. The computer-assisted surgical system 200 generally includes an optical tracking system 206 in communication with a surgical workflow, a display monitor 212 having a graphical user interface (GUI) for displaying one or more steps of the surgical workflow, and two or more trackable devices (e.g., a tracked digitizer probe 230 and a tracked surgical device 204). More specifically, the optical tracking system 206 includes two or more optical cameras having a field of view (FOV) of the surgical site to permit the tracking system 206 to track one or more of the trackable devices in the FOV. In particular embodiments, the optical cameras are positioned in the operating room (OR) to limit the FOV of the surgical site, intentionally or not. For example, the optical cameras may be positioned inside a surgical lamp situated above the surgical site. In which case, the FOV of the surgical site is limited because the surgical lamp is pointed directly at the site of operation (e.g., a FOV of just one or two bones). This is in contrast to other surgical systems where the optical tracking system is situated in the OR to cover a large FOV to track many, if not all, of the trackable devices contemporaneously.

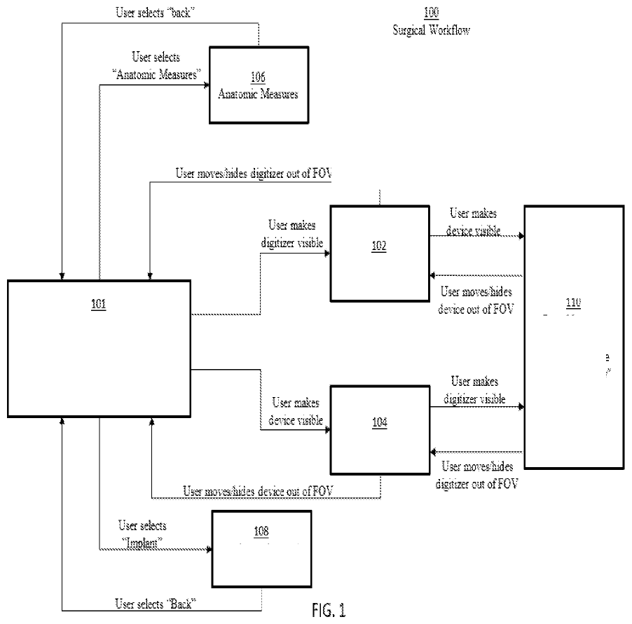

With reference now to the figures, FIG. 1 illustrates a flowchart of a high-level surgical workflow 100 for performing a computer-assisted orthopedic procedure. The surgical workflow 100 includes a plurality of steps, including a first step displaying a main menu 101, a second step being a registration mode 102 having instructions for registering one or more bones, a third step being a surgery mode 104 having instructions to perform one or more actions on one or more bones, a fourth step being an anatomic measures mode 106 for acquiring measurements of the anatomy, and a fifth step being an implant selection/finalize surgery mode 108 to finalize the surgery. Each step may further include sub-steps as further described below. In addition, it should be appreciated that the terms first, second, third, etc. do necessarily refer to a sequential order but rather identify different steps in the workflow 100.

The surgical workflow 100 begins with the main menu 101. The main menu 101 provides the user with several available options including: 1) bring a tracked digitizer probe 230 in the FOV of the tracking system; 2) bring a tracked surgical device 204 in the FOV of the tracking system; 3) select “Anatomic Measures” on the GUI; and 4) select “Implant/Finalize” on the GUI. The main menu 101 is initially displayed to the user once one or more pre-surgical steps (calibration, diagnostics, and set-up) have been completed. The user may then execute one of the available options. The main menu 101 may also include an option for a user to view the FOV as seen from the optical cameras on the GUI in order for the user to fully grasp the boundaries of the FOV before or during a procedure. Additionally, a “FOV Perspective” option may be provided on the GUI at all times during a procedure for a user to pull up a picture in picture view the FOV as seen from the optical cameras. Similarly, a “FOV projection” option may be included on the main menu 101 and/or on the GUI at all times, which when activated projects a colored light, for example a red light, from the area of the surgical lamp or optical cameras onto the area of the procedure or operation, such that a user is able to visualize the boundaries of the FOV of the optical cameras while interfacing with the system and performing the method.

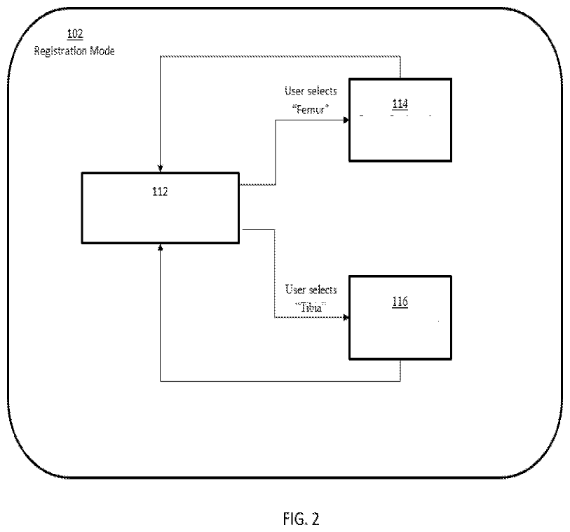

The registration mode 102 is displayed when the user introduces only the tracked digitizer probe 230 into the FOV. More particularly, when the user introduces only the tracked digitizer probe 230 in the FOV, the optical tracking system performs the following: a) identifies the tracked digitizer probe 230 based on a reference member (e.g., an attached tracking array 220c or fiducial markers 330 having a unique geometry, a unique emitting wavelength, or a unique emitted signal) associated with the digitizer probe 230; b) determines which step in the surgical workflow 100 utilizes the tracked digitizer probe 230; and c) commands the workflow to display the registration mode 102 to the user on the GUI. If the user then removes or hides the digitizer probe 230 from the FOV, then the workflow 100 returns to the main menu 101, with one caveat. In the event the user initiated a registration procedure in the registration mode 102 (by for example selecting a bone to register), then removal of the digitizer probe 230 from the FOV does not cause the workflow 100 to divert to the main menu 101, but rather the registration mode 102 stays active until the user completes registration of at least one bone. Therefore, if the digitizer probe 230 becomes hidden from the FOV of the tracking system 206 after the registration has been initiated, the registration process is not prematurely and automatically aborted. Further details of the registration mode 102 are provided below with reference to FIG. 2.

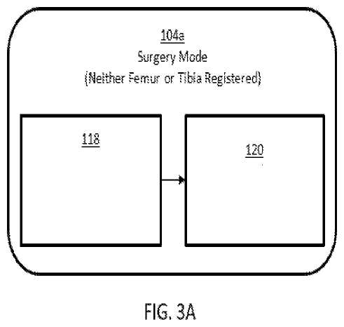

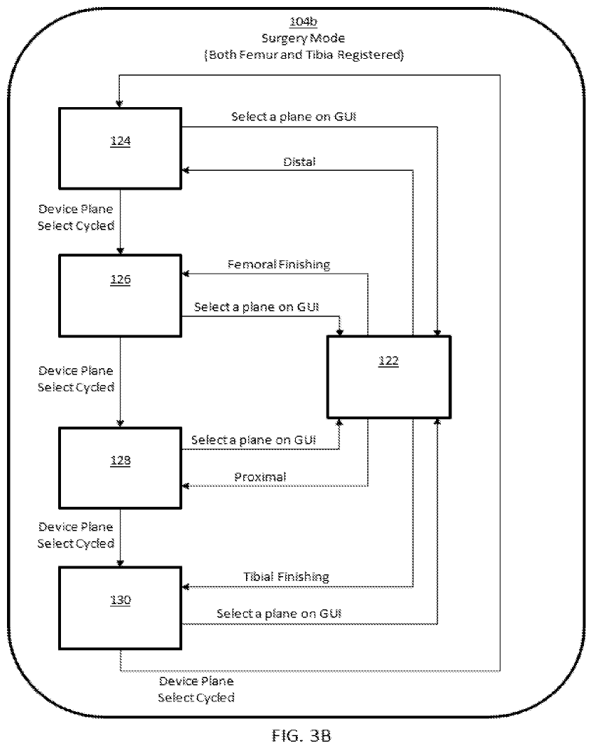

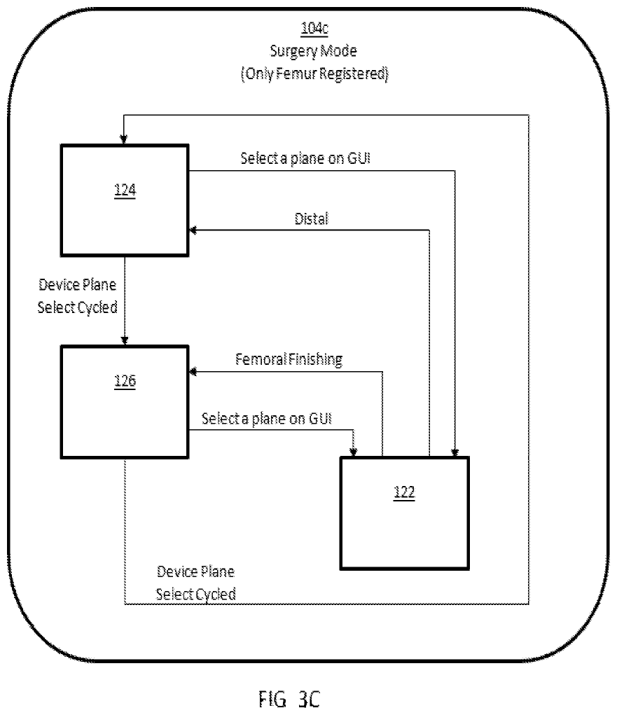

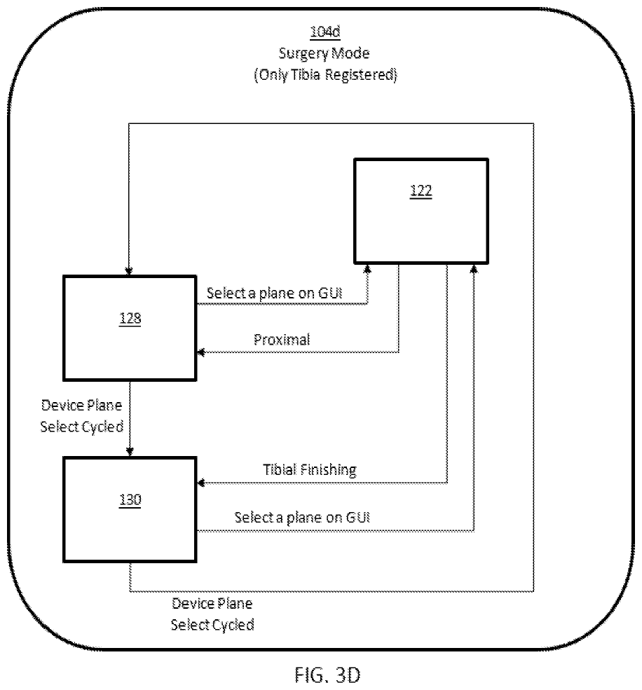

The surgery mode 104 is displayed when the user introduces only the tracked surgical device 204 into the FOV. More particularly, when the user introduces only the tracked surgical device 204 in the FOV, the optical tracking system 206 does the following: a) identifies the tracked surgical device 204 based on a reference member (e.g., an attached tracking array or fiducial markers 314 having a unique geometry, a unique emitting wavelength, or a unique emitted signal) associated with the surgical device 204; b) determines which step in the surgical workflow 100 utilizes the surgical device 204; and c) commands the workflow to display the surgery mode 104 to the user on the GUI. At any time, if the user removes or hides the tracked surgical device 204 from the FOV, then the workflow 100 returns to the main menu 101. Further details of the surgery mode 104 are further described below with reference to FIGS. 3A-3D.

In a particular inventive embodiment, in the event the tracked digitizer probe 230 and the tracked surgical device 204 are in the FOV contemporaneously, then an error message 110 is displayed on the GUI. The error message 110 instructs the user to remove either the digitizer 230 or the surgical device 204 from the field of view. Therefore, the tracking system 206 can determine which step to display in the workflow 100 based on the intention of the user. Once, either the tracked digitizer probe 230 or surgical device 204 is removed from the FOV, the workflow 100 displays the proper step (i.e., registration mode 102 or surgery mode 104). If both devices are removed from the FOV, then workflow 100 displays the main menu 101.

The anatomic measures mode 106 is accessed when a user selects the anatomic measures option on the GUI from the main menu 101. The anatomic measures mode 106 permits the user to choose between the following options: a) flexion-extension range of motion; b) varus-valgus laxity; and c) limb alignment. The flexion-extension option is available any time after the patient has been prepared (e.g., the bone(s) are exposed but not necessarily registered), the varus-valgus laxity is available only after one bone has been registered, and the limb alignment is available only after both bones have been registered. The anatomical measurements provide the user with intra-operative measurements to adjust or verify the bone cuts and implant positioning on the bone. In more detail, in the flexion-extension measurement option, the workflow 100 displays a lateral view of a limb on the GUI. The positions of a first tracking array installed on a first bone (e.g., femur F) and a second tracking array installed on a second bone (e.g., tibia T) are tracked through flexion, and the lateral view is updated to match the current relative position of both bones. If neither bone has been registered, the workflow 100 displays the range of flexion only after sufficient flexion has been performed for the application to estimate the positions of the bones relative to the markers. If both bones have been registered, then the workflow 100 displays the range of flexion immediately, and also displays the maximum flexion angle and maximum extension angle using the mechanical axes of the first bone and the second bone defined in a pre-operative plan generated in a pre-operative planning workstation. The user also has the option to reset the measured range of flexion. In the varus-valgus measurement option, the workflow 100 displays a coronal view of a limb on the GUI. The positions of the first tracking array and the second tracking array are likewise tracked through motion in the coronal plane, and updates the coronal view to match the current relative position of both bones. If only one bone has been registered, then the workflow 100 displays the range of varus-valgus laxity. If both bones have been registered, then the workflow 100 displays the range of varus-valgus motion and also displays the maximum valgus laxity and the maximum varus laxity using the mechanical axes of the first bone and the second bone defined in the pre-operative surgical plan. The user likewise has the option to reset the measured range of varus-valgus laxity. In the limb alignment measurement option, the workflow 100 displays a coronal view of a limb on the GUI. The position of the first tracking array and the second tracking array are used to display the current limb alignment using the mechanical axes of the first bone and second bone as defined in the pre-operative surgical plan.

The implant selection/finalize surgery mode 108 is accessed when a user selects said option on the GUI from the main menu 101. During the finalize surgery step 108, the workflow 100 displays information regarding the planned implants. The anatomic measures mode 106 is also accessible from the finalize surgery mode step 108. Pressing a “complete” button on the GUI progresses the workflow 100 to display instructions to remove the tracking arrays from the patient. An option in the finalize surgery mode 108 also permits the user to return to the main menu 101. Once the user finalizes the surgery, the surgery is complete, the data is archived, and any other instructions to wrap-up post-surgery is displayed on the GUI (e.g., remove tracking arrays, tear-down instructions, sterilization instructions).

With reference now to FIG. 2, the registration mode 102 is shown in more detail, where the registration mode 102 guides a user in registering a surgical plan or surgical planning data to one or more bones. The registration mode 102 includes a registration mode menu 112 instructing the user to select a bone to register. In specific embodiments, the user selects either the femur F or tibia T. The tracked digitizer probe 230 may include two or more buttons (334a, 334b) in optical communication with the tracking system 206 to aid in the selection, where each button (334a, 334b) corresponds to the femur F and the tibia T, respectively. Once the user selects either the femur F or tibia T, the workflow 100 is directed to either a femur registration module 114 or a tibia registration module 116. In the femur registration module 114 an image of the distal femur with a plurality of registration points for collection is displayed on the GUI. Likewise, in the tibia registration module 116 an image of the proximal tibia with a plurality of registration points for collection is displayed on the GUI. The user then collects each of the displayed points on the femur F or tibia T by placing the probe tip 336 on the bone at the designated location and clicking one of the buttons (334a, 334b). According to some inventive embodiments, the tracked digitizer probe 230 may include a feedback mechanism, such as lights, a speaker, or a vibrating element, that activates when a user either is moving the tracked digitizer probe 230 closer to each of the plurality of registration points for collection or when the user has successfully registered a registration point, to ensure accuracy in registration point collection. The user may delete a point by holding one or more of the buttons (334a, 334b) for a pre-determined time if needed. Some inventive embodiments include condition monitoring of the probe tip 336, and the system alerts the user on the GUI if the condition falls below a predetermined threshold. When the condition is determined to fall below the predetermined threshold, the workflow control system instructs a user to recondition the tip, providing instructions for such reconditioning. Once the registration is successful, the transformed surgical planning data (e.g., the location of one or more target planes relative to the bone, operational data to control the surgical device 204, and/or POSE data of the bone(s) and the surgical device 204 from the tracking system) is optically transmitted to the tracked surgical device 204 when the tracked surgical device 204 is in the FOV. The surgical planning data for one bone may be transmitted independent of surgical planning data for a second bone to permit the user to register and prepare just one bone if desired. In specific inventive embodiments, after the surgical planning data is transmitted to the surgical device 204, one or more surgical steps in the surgery mode 104 are automatically selected. For example, if only the femur F is registered, the system automatically selects a femoral distal cut. If only the tibia T is registered, the system automatically selects a tibial proximal cut, and if both the femur F and tibia T are registered then the system automatically selects the femoral distal cut.

With reference now to FIGS. 3A-3D, four potential surgery modes (104a, 104b, 104c, 104d) in the workflow 100 are shown depending on the registration status of the bones. FIG. 3A depicts a first surgery mode 104a when neither the femur nor tibia are registered. The user is simply instructed to remove or hide the surgical device 204 from the FOV and to register at least one bone. The workflow 100 then returns to the main menu 101 upon the hiding or removal of the surgical device 204. FIG. 3B depicts a second surgery mode 104b having a workflow when both the femur F and tibia T are registered. The system determines which surgical action to perform on a bone based on input from either a selection input mechanism 322 located directly on the tracked surgical device 204, or from a selection made on the GUI. In particular embodiments, the surgical action involves inserting pins on a target plane as described in PCT Int. App. No. US2016/062020, now U.S. patent Ser. No. 15/778,811 assigned to the assignee of the present application and incorporated by reference herein. The system then determines which plane is selected 122 and based on which plane is selected, the system displays instructions to complete that surgical action for that plane including a list of required accessories (e.g., cut guides, bone pins). The instructions remain on the GUI until: a) a new plane is selected (either “select a new plane on GUI”, or with selection input mechanism 322 on the surgical device 204 (i.e., “Device Plane Select Cycled”)); b) a different trackable device enters the FOV (error message); or c) the user chooses another available option on the GUI. Specifically for the 2-DOF articulating surgical device 204, the available cut planes include the distal cut 124, femoral finishing 126, proximal cut 128, and Anterior-Posterior (A-P) line 130 also referred to as tibial finishing. The distal cut 124 provides instructions for inserting pins to receive a cut guide to create the distal cut plane on the distal femur. The femoral finishing 126 provides instructions for inserting pins on the distal cut plane to receive a cut guide to create the remaining femoral cuts (e.g., anterior cut plane, posterior cut plane, anterior chamfer cut plane, and posterior chamfer cut plane). The proximal cut 128 provides instructions for inserting pins to create the proximal cut plane on the tibia. And, the A-P line 130 provides instructions for marking internal-external rotation for the tibial component. FIG. 3C depicts a third surgery mode 104c when only the femur is registered and therefore only includes the femur workflow instructions from the second surgery mode 104b. FIG. 3D depicts a fourth surgery mode 104d when only the tibia is registered and therefore only includes the tibia workflow instructions from the second surgery mode 104b. Some inventive embodiments include condition monitoring of the surgical device 204, particularly a tool 306 of the surgical device 204, and the system alerts the user on the GUI if the condition falls below a predetermined threshold for the tool 306. When the condition is determined to fall below the predetermined threshold, the workflow control system instructs a user to recondition the tool, providing instructions for such reconditioning.

### Computer-Assisted Surgical System

The aforementioned workflow 100 is implementable with a variety of different computer-assisted surgical systems and surgical procedures. Examples of computer-assisted surgical systems include a tracked 1-N degree of freedom hand-held surgical system, a tracked autonomous serial-chain manipulator system, a tracked haptic serial-chain manipulator system, a tracked parallel robotic system, or a master-slave robotic system, as described in U.S. Pat. Nos. 7,206,626, 8,876,830, and 8,961,536, 9,566,122, U.S. Pat. App. No. 2013/0060278, and PCT Intl. App. No. US2016/062020 all of which are incorporated by reference herein in their entireties.

With reference to FIG. 4 and FIG. 5, a specific inventive embodiment of a 2-degree-of-freedom (2-DOF) surgical system 200 is shown, which is described in more detail in PCT Intl. App. No. US2016/062020 assigned to the assignee of the present application. The 2-DOF surgical system 200 generally includes a computing system 202, an articulating surgical device 204, and a tracking system 206. The surgical system 200 is able to guide and assist a user in accurately placing pins coincident with a target pin plane that is defined relative to a subject's bone. The target plane is defined in a surgical plan and the pins permit the assembly of various cut guides and accessories to aid the surgeon in making the cuts on the femur and tibia to receive a prosthetic implant in a planned POSE.

With reference to FIG. 5, the components of the articulating surgical device 204 is shown in more detail. The surgical device 204 includes a hand-held portion 302 and a working portion 304. The hand-held portion 302 includes an outer casing of ergonomic design to be held and manipulated by a user. The working portion 304 includes a tool 306 having a longitudinal tool axis. The tool 306 is driven by a motor 305 and attached thereto with a chuck 307. A trigger 309 may activate the motor 305 and permit other user inputs. The hand-held portion 302 and working portion 304 are connected by a front transmission assembly 308a and a back transmission assembly 308b that adjust the pitch and translation of the working portion 304 relative to the hand-held portion 302. Each transmission assembly (308a, 308b) includes a linear rail, a linear guide, a ball nut, and a ball screw. A first end of each linear rail is attached to the working portion 304 via a hinge (310a, 310b), where the hinges (310a, 310b) allow the working portion 304 to pivot relative the transmission assemblies (308a, 308b). The ball nuts are attached at opposing ends of the linear rails and are in mechanical communication with the ball screws. A front ball screw is driven by a front actuator 312a and a rear ball screw is driven by a rear actuator 312b. The actuators (312a, 312b) may be servo-motors that bi-directionally rotate the ball screws. The actuators (312a, 312b) power the ball screws which cause the ball nuts, and therefore the linear rails, to translate along the axis of the ball screws. Accordingly, the translation and pitch of the working portion 304 may be adjusted depending on the position of each ball nut on their corresponding ball screw. A linear guide 222 may further constrain and guide the motion of the linear rails in the translational direction.

The articulating device 302 further includes three or more fiducial markers (314a, 314b, 314c, 314d), rigidly attached to or incorporated into to the working portion 304 to permit a tracking system 206 to track the POSE of the working portion 304. The fiducial markers (314a, 314b, 314c, 314d) may be active markers such as light emitting diodes (LEDs), or passive markers such as retroreflective spheres. The three or more fiducial markers (314a, 314b, 314c, 314d) act as the reference member associated with the surgical device 204 that permits the tracking system 206 to identify the surgical device 204 in the FOV. The three or more fiducial markers (314a, 314b, 314c, 314d) may uniquely identify the surgical device 304 based on either a unique geometry of the markers (314a, 314b, 314c, 314d), or a unique emitted wavelength/frequency of the markers (314a, 314b, 314c, 314d). In a specific inventive embodiment, the surgical device 304 further includes an optical communications modem 316 to provide a serial interface to relay data and commands between the surgical device ‘host’ processors (e.g., electronics module 320 described below) and other subsystems such as the optical tracking system 206 or a navigation computer 208. The optical communications modem 316 may emit data via a dedicated infrared LED 316 and receive data via a photodiode 318. The surgical device 304 may further include a removable battery and electronics module 320 which control the actuators (312a, 312b). The electronics module 320 includes a microcontroller to provide local state control, and implements most of the actuator control functionality. The microcontroller communicates with other subsystems (e.g., optical tracking system 206, navigation computer 208, and workflow 100) via the optical communications modem 316. Data transactions include: a) receiving target planes from the computing system 202 to the surgical device 204; b) receiving real time marker POSEs from the optical tracking system 206; c) sending status/acknowledge packets from the surgical device 204 to the tracking system 206 or navigation computer 208 (e.g., battery voltage, target plane selection; fault conditions); and d) uploading data logs from the surgical device 204.

The surgical device 204 further includes a plane selection input mechanism 322, a plurality of feedback selection LEDs (324a, 324b, 324c, 324d), and a power/status LED 326. The plane selection input mechanism 322 may include one or more buttons, or sliding toggle, to permit the user to select one or more of the planes as described above (i.e., distal cut 124, femoral finishing 126, proximal cut 128, A-P line 130). The feedback selection LEDs (324a, 324b, 324c, 324d) indicates to the user which plane is selected. The power/status LED relays status information. For example, if there is no power to the device, the LED is off. Flashing green may indicate there is power but surgical planning data has not been downloaded. Solid green means ready for use. Flashing amber to indicate a low battery and solid red for a hardware fault.

With reference back to FIG. 4, the computing system 202 in some inventive embodiments includes: a navigation computer 208 including a processor; a planning computer 210 including a processor; a tracking computer 211 including a processor, and peripheral devices. Processors operate in the computing system 202 to perform computations associated with the inventive system and method. It is appreciated that processor functions are shared between computers, a remote server, a cloud computing facility, or combinations thereof.

In particular inventive embodiments, the navigation computer 208 may include one or more processors, controllers, and any additional data storage medium such as RAM, ROM or other non-volatile or volatile memory to perform functions related to controlling the surgical workflow 100 and provide guidance to the user, interpret pre-operative planning surgical data, and communicating the target plane positions to the surgical device 204. In some embodiments, the navigation computer 208 is in direct communication with the optical tracking system 206 such that the optical tracking system 206 may identify trackable devices in the FOV and the navigation computer 208 can control the workflow 100 accordingly based on the identity of the tracked device. However, it should be appreciated that the navigation computer 208 and the tracking computer 211 may be separate entities as shown, or it is contemplated that their operations may be executed on just one or two computers depending on the configuration of the surgical system 200. For example, the tracking computer 211 may have operational data to directly control the workflow 100 without the need for a navigation computer 208. Or, the navigation computer 208 may include operational data to directly read data detected from the optical cameras without the need for a tracking computer 211. In any case, the peripheral devices allow a user to interface with the surgical system 200 and may include: one or more user interfaces, such as a display or monitor 212; and various user input mechanisms, illustratively including a keyboard 214, mouse 222, pendent 224, joystick 226, foot pedal 228, or the monitor 212 may have touchscreen capabilities.

The planning computer 210 is preferably dedicated to planning the procedure either pre-operatively or intra-operatively. For example, the planning computer 210 may contain hardware (e.g. processors, controllers, and memory), software, data, and utilities capable of receiving and reading medical imaging data, segmenting imaging data, constructing and manipulating three-dimensional (3D) virtual models, storing and providing computer-aided design (CAD) files, planning the POSE of the implants relative to the bone, generating the surgical plan data for use with the system 200, and providing other various functions to aid a user in planning the surgical procedure. The planning computer also contains software dedicated to defining target planes. The final surgical plan data may include an image data set of the bone, bone registration data, subject identification information, the POSE of the implants relative to the bone, the POSE of one or more target planes defined relative to the bone, and any tissue modification instructions. The final surgical plan is readily transferred to the navigation computer 208 and/or tracking computer 211 through a wired or wireless connection in the operating room (OR); or transferred via a non-transient data storage medium (e.g. a compact disc (CD), a portable universal serial bus (USB drive)) if the planning computer 210 is located outside the OR. The registered surgical planning data is then optically transmitted to the surgical device 204 as described above.

In a particular embodiment, the tracking system 206 is an optical tracking system as described in U.S. Pat. No. 6,061,644, having two or more optical camera (not shown because the cameras are situated inside a surgical lamp 218 and directed towards the surgical site) to detect the position of fiducial markers arranged on rigid bodies (tracking arrays) or integrated directly into the tracked devices. Illustrative examples of the fiducial markers include: an active transmitter, such as an LED or electromagnetic radiation emitter; a passive reflector, such as a plastic sphere with a retro-reflective film; or a distinct pattern or sequence of shapes, lines or other characters. A set of fiducial markers arranged on a rigid body is referred to herein as a tracking array (220a, 220b, 220c), however, the fiducial markers may be integrated directly into the tracked devices. Each fiducial marker array (220a, 220b, 220c) or set of fiducial markers on each tracked device has a unique geometry/arrangement of fiducial markers, or a unique transmitting wavelength/frequency if the markers are active LEDS, such that the tracking system 206 can distinguish between each of the tracked objects and therefore act as the reference members associated with each tracked device.

In specific inventive embodiments, the tracking system 106 is built into a surgical lamp 218, which therefore limits the FOV of the optical cameras. However, in other embodiments the tracking system 206 and cameras are located on a boom, stand, or built into the walls or ceilings of the operating room. The tracking system computer 211 includes tracking hardware, software, data, and utilities to determine the POSE of objects (e.g. bones such as the femur F and tibia T, the surgical device 204) in a local or global coordinate frame. The POSE of the objects is referred to herein as POSE data, where this POSE data is readily communicated to the navigation computer 208 and the electronics modules 320 through a wired or wireless connection.

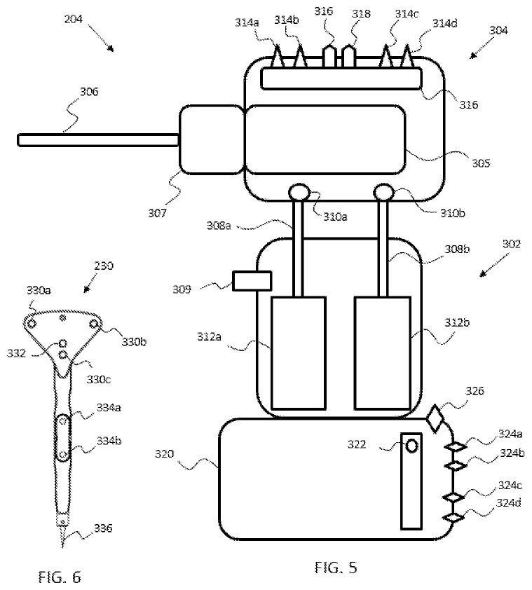

The surgical system 200 further includes a tracked digitizer probe 230 as mentioned above for registering one or more bones. With reference to FIG. 6, a detailed view of the tracked digitizer probe 230 is shown. The tracked digitizer probe 230 include three or more fiducial markers (330a, 330b, 330c), an optical communications LED 332, two or more selection buttons (334a, 334b), and a probe tip 336. The fiducial marker arrays (330a, 330b, 330c) may be present on a tracking array 220c or directly incorporated into the probe 230 in a unique fashion to permit the tracking system 206 to identify the tracked digitizer probe 230. The optical communications LED 332 allows the probe 230 to communicate with the tracking system 206 and/or navigation computer 208. The two or more selection buttons (334a, 334b) allows the user to select between the femur and tibia in the registration mode menu 112 as described above. The buttons (334a, 334b) also allows the user to click and collect a point during the registration procedure.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method for controlling a workflow during a computer-assisted surgical procedure, comprising:
providing an optical tracking system having a field of view of a surgical site, said optical tracking system in communication with the workflow;
introducing a first tracked device into said field of view;
identifying said first tracked device with said optical tracking system based on a first reference member associated with said first tracked device;
determining a first step in the workflow based on the identifying of the first tracked device; and
displaying said first step to a user on a graphical user interface.

2. The method of claim 1 further comprising:
introducing a second tracked device while said first tracked device is in said field of view contemporaneously; and
displaying an error message to said user on said graphical user interface to remove at least one of said first tracked device or second tracked device from said field of view.

3. The method of claim 2 further comprising:
removing said first tracked device from said field of view;
introducing said second tracked device into said field of view;
identifying said second tracked device based on a second reference member associated with said second tracked device;
determining a second step in the workflow based on the identifying of said second tracked device; and
displaying said second step to a user on a graphical user interface.

4. The method of claim 3 further comprising:
displaying a main menu of the workflow to the user on said graphical user interface when neither said first tracked device is in said field of view and said second tracked device is in said field of view.

5. The method of claim 4 wherein said first step and said second step are either sequential or non-sequential.

6. The method of claim 4 wherein said first tracked device is a tracked digitizer probe and said second tracked device is a tracked surgical device.

7. The method of claim 6 wherein said step in the workflow includes instructions to register a bone with said tracked digitizer probe.

8. The method of claim 6 further comprising:
requesting user acknowledgement to initiate bone registration when only said tracked digitizer probe is in said field of view;
acknowledging said requesting; and
registering the bone, wherein removal of said tracked digitizer probe from said field of view does not cause the workflow to display said main menu until said registration is complete.

9. The method of claim 6 wherein said second step in the workflow includes instructions to perform at least one action on the bone with said tracked surgical device.

10. The method of claim 9 wherein said second step in the workflow comprises a first surgery mode, a second surgery mode, a third surgery mode, and a fourth surgery mode.

11. The method of claim 9 wherein said at least one action performed on the bone is at least cutting the bone.

12. The method of claim 10 further comprising:
initiating said first surgery mode when no bone is registered and instructing the user via said graphical user interface to remove or hide said tracked surgical device from said field of view;
initiating said second surgery mode when a first bone and a second bone are registered and providing instructions via said graphical user interface to perform at least one action on the first bone and the second bone;
initiating said third surgery mode when only the first bone is registered and providing instruction via said graphical user interface to perform at least one action on only the first bone; and
initiating said fourth surgery mode when only the second bone is registered and providing instruction via said graphical user interface to perform at least one action on only the second bone.

13. The method of claim 12 wherein the first bone is a femur and the second bone is a tibia.

14. The method of claim 6 wherein said tracked surgical device is a 2-degree-of-freedom articulating surgical device to insert pins in the femur and tibia according to a surgical plan.

15. The method of claim 6 wherein said tracked surgical device is at least a 5-degree-of-freedom surgical robot to actively remove material from the femur or tibia according to a surgical plan.

16. A computer-assisted surgical system, comprising:
an optical tracking system having a field of view of a surgical site, said tracking system in communication with a surgical workflow having a plurality of steps displayable on a graphical user interface;
a tracked digitizer probe; and
a tracked surgical device;
wherein said optical tracking system includes a processor having software executable instructions that when executed by said processor causes said processor to:
identify presence or absence of either said tracked digitizer probe or tracked surgical device in said field of view of said tracking system;
determine a step in the workflow based on said identification of either said tracked digitizer probe or tracked surgical device; and
command the workflow to display said determined step on said graphical user interface.

17. The system of claim 16 further comprising executable instructions that when executed by said processor causes said processor to:
identify both said tracked digitizer probe and tracked surgical device in said field of view contemporaneously; and
command the workflow to display an error message on said graphical user interface instructing the user to remove at least one of said tracked digitizer probe or tracked surgical device from said field of view.

18. The system of claim 17 wherein said tracked surgical device is a 2-degree-of-freedom articulating surgical device to insert one or more pins in a bone.

19. The system of claim 18 further comprising a surgical plan having a plurality of target planes defined relative to a bone model therein, said target planes designating a position to insert one or more pins in the bone with said 2-degree-of-freedom articulating surgical device.

20. The system of claim 19 wherein said articulating surgical device includes a switch to permit a user to switch between said plurality of virtual planes.

21. (canceled)
